(19) United States
(12) Patent Application Publication
(10) Pub. No.: US 2024/0033307 A1
(43) Pub. Date: Feb. 1, 2024

(54) METHOD FOR ENHANCING SPORTS PERFORMANCE AND ALLEVIATING SARCOPENIA WITH STREPTOCOCCUS THERMOPHILES ST7 FERMENTATION PRODUCT COMPOSITION

(71) Applicant: SYNGEN BIOTECH CO., LTD., Tainan City (TW)

(72) Inventors: Wei-Jen CHEN, Tainan City (TW); Tsung-Yin TANG, Tainan City (TW); Ming-Chung TSENG, Tainan City (TW); Yu-Shan WEI, Tainan City (TW)

(21) Appl. No.: 18/071,215

(22) Filed: Nov. 29, 2022

(30) Foreign Application Priority Data
Jul. 29, 2022 (TW) 111128691

(51) Int. Cl. A61K 35/744; A61K 31/7084; A61P 21/06

(52) U.S. Cl. CPC A61K 35/744; A61K 31/7084; A61P 21/06

(57) ABSTRACT

A method for enhancing sports performance and alleviating sarcopenia with a Streptococcus thermophiles ST7 fermentation product composition is disclosed. The administration of an effective amount of the Streptococcus thermophiles ST7 fermentation product composition to an individual in advance can increase the ATP content of the individual's muscle cells and lower indices related to fatigue and muscle damage, thereby enhancing the individual's sports performance and improving or preventing sarcopenia and its symptoms.

### BACKGROUND OF THE INVENTION

### 1. Technical Field

The present invention relates to uses of probiotics. More particularly, the invention relates to a method for enhancing sports performance and alleviating sarcopenia with a Streptococcus thermophiles ST7 fermentation product composition.

### 2. Description of Related Art

The rapid advancement of medical care and technology has increased, and keeps increasing, the human lifespan. Many countries, therefore, are facing the health issues of an aging or aged society. One such issue is sarcopenia, which is considered a major factor in deterioration of the health of the elderly. Sarcopenia refers to a group of symptoms identifiable with a combination of measurement indices such as muscle mass, muscle strength, and muscle function, and is believed to be associated with disabilities, falls, and an increase in the death rate.

Currently, there is no drug for treating sarcopenia clinically. The symptoms of sarcopenia can only be improved by adjusting a patient's eating habits and exercise habits to reduce further deterioration. However, as most patients diagnosed with sarcopenia already have such symptoms as slowness in motion, low grip strength, and muscle weakness, it is not uncommon that a patient with sarcopenia gives up an ongoing exercise therapy because of fatigue or muscle soreness, and this adds to the difficulty of ameliorating sarcopenia.

### BRIEF SUMMARY OF THE INVENTION

The primary objective of the present invention is to provide uses of a Streptococcus thermophiles ST7 fermentation product composition in enhancing sports performance and alleviating sarcopenia, i.e., to provide administration of an effective amount of the Streptococcus thermophiles ST7 fermentation product composition to an individual to increase the adenosine triphosphate (ATP) content and glycogen content of the individual's muscle cells and lower biochemical indices related to fatigue and to muscle tissue damage, the goal being to enhance the individual's sports performance effectively (e.g., reducing muscle fatigue, accelerating the removal of lactic acid from muscles after exercise, and enhancing motor ability) and to improve or alleviate sarcopenia and its symptoms.

To achieve the foregoing objective, the present invention provides uses of Streptococcus salivarius subsp. thermophilus ST7 (or Streptococcus thermophiles ST7 for short) and of a fermentation product thereof. The fermentation product of the Streptococcus salivarius subsp. thermophilus ST7 (or the Streptococcus thermophiles ST7 fermentation product for short) is obtained by fermentation of the Streptococcus salivarius subsp. thermophilus ST7 and includes viable bacteria of the Streptococcus salivarius subsp. thermophilus ST7, inactivated bacteria of the Streptococcus salivarius subsp. thermophilus ST7, metabolites of the Streptococcus salivarius subsp. thermophilus ST7, or NADH (nicotinamide adenine dinucleotide in reduced form). The Streptococcus salivarius subsp. thermophilus ST7 is deposited at the Food Industry Research and Development Institute, Hsinchu, Taiwan (the deposit date being Apr. 25, 2022, and the deposit number being BCRC 911126) and at German Collection of Microorganism Cell Cultures (DSMZ) (the deposit date being Apr. 28, 2022, and the deposit number being DSM 34255).

The embodiments provided herein of the present invention disclose using the Streptococcus salivarius subsp. thermophilus ST7 and/or its fermentation product to prepare a composition for relieving muscle fatigue, enhancing an individual's sports performance, or treating sarcopenia. An effective amount of the Streptococcus salivarius subsp. thermophiles ST7 and/or its fermentation product can be administered to an individual to increase the ATP content of the individual's muscle cells, to increase the glycogen content of muscle cells and liver cells, to lower the blood lactic acid content and blood urea nitrogen (BUN) concentration, and to increase the repair ability of muscle cells, thereby making a significant improvement in the individual's sports performance in terms of endurance, grip strength, and sustained physical exertion, and alleviating or improving sarcopenia and its symptoms.

The individual may belong to a population at high risk of sarcopenia.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

FIG. 1A shows the analysis results of an NADH standard derivatized with acetophenone, with the graph on the left being a high-performance liquid chromatography (HPLC) chromatogram (382 nm), and the graph on the right being a photodiode-array (PDA) absorbance spectrum.

FIG. 1B shows the analysis results of the Streptococcus thermophiles ST7 fermentation product composition disclosed herein derivatized with acetophenone, with the graph on the left being an HPLC chromatogram (382 nm), and the graph on the right being a PDA absorbance spectrum.

FIG. 2 is a spectrum showing the electrospray ionization mass spectrometry (ESI-MS) analysis result of the Streptococcus thermophiles ST7 fermentation product composition disclosed herein.

FIG. 3 shows the ATP contents of differently treated C2C12 myoblasts as quantified with an ATP colorimetric assay kit.

FIG. 4 shows the observation results about the death of differently treated C2C12 myoblasts, with “+” indicating staining with crystal violet, and “−” indicating no staining with crystal violet.

### DETAILED DESCRIPTION OF THE INVENTION

The present invention discloses uses of a Streptococcus thermophiles ST7 fermentation product composition in enhancing sports performance and alleviating sarcopenia. The Streptococcus thermophiles ST7 fermentation product composition disclosed herein includes Streptococcus salivarius subsp. thermophilus ST7 and/or a fermentation product obtained by fermentation of the Streptococcus salivarius subsp. thermophilus ST7. Continuous administration of an effective amount of the Streptococcus thermophiles ST7 fermentation product composition to an individual in advance can increase the ATP content of the individual's muscle cells, enhance the individual's muscle abilities, lower the degree of muscle fatigue, and promote the metabolism of lactic acid and the repair of damaged muscles, thereby enhancing the individual's sports performance and improving or preventing sarcopenia and its symptoms.

The “Streptococcus thermophiles ST7 fermentation product composition” disclosed herein includes an effective amount of the Streptococcus salivarius subsp. thermophilus ST7 and/or a fermentation product thereof. The term “effective amount” refers to a dose that allows the Streptococcus salivarius subsp. thermophilus ST7 and/or its fermentation product to be active to an organism as can be understood in the field to which the present invention pertains, such as 1%-100%. The Streptococcus salivarius subsp. thermophilus ST7 may be viable bacteria or inactivated bacteria. The Streptococcus thermophiles ST7 fermentation product composition may be prepared as needed as food, a nutritional supplement, a pharmaceutical, or an adjuvant; may be added with a drug carrier, an excipient, a flavoring agent, and so on that are safe to the human body; and may be prepared in the dosage form of tablets, powder, solution, suspension, or other appropriate dosage forms.

The “fermentation product” disclosed herein refers to a product obtained by fermentation of the Streptococcus salivarius subsp. thermophilus ST7 and subsequent cultivation. The step of collecting the fermentation product may be carried out by centrifugation, filtration, direct condensation, drying, or other methods, and the constitution of the fermentation product may vary with the method of the collection step. More specifically, the fermentation product at least includes viable bacteria of the Streptococcus salivarius subsp. thermophilus ST7, inactivated bacteria of the Streptococcus salivarius subsp. thermophilus ST7, metabolites of the Streptococcus salivarius subsp. thermophilus ST7, or NADH. For example, if the collection process is performed in such a way that the fermentation product is directly dried by heating, without being filtered in advance, the fermentation product will at least include inactivated bacteria of the Streptococcus salivarius subsp. thermophilus ST7, metabolites of the Streptococcus salivarius subsp. thermophilus ST7, and NADH.

The “Streptococcus salivarius subsp. thermophilus ST7” disclosed herein is deposited at the Food Industry Research and Development Institute, Hsinchu, Taiwan (the deposit date being Apr. 25, 2022, and the deposit number being BCRC 911126) and at German Collection of Microorganism Cell Cultures (DSMZ) (the deposit date being Apr. 28, 2022, and the deposit number being DSM 34255). The Streptococcus salivarius subsp. thermophilus ST7 can be cultivated in a common growing culture medium, such as a culture medium containing 1%-2% glucose, 1%-2% peptone, and 0.01%-0.08% magnesium sulfate, among other ingredients. In addition, the Streptococcus salivarius subsp. thermophilus ST7 disclosed herein can produce NADH, so the product obtained by fermentation of the Streptococcus salivarius subsp. thermophilus ST7 as a starter and by subsequent cultivation includes NADH.

The term “grip strength” is used herein in relation to an animal test and, when applied to the human body, refers to gripping force. The gripping force is a major index for diagnosing sarcopenia. If a substance can increase an individual's grip strength or gripping force, then the substance is helpful in improving sarcopenia.

To shed light on the technical features and effects of the present invention, a number of experiments are detailed below with reference to the accompanying drawings.

All the cells (cell lines) used in the experiments are easily available to a person of ordinary skill in the art and therefore are not required to be deposited.

The numerical data of the experiments is expressed as Mean±SD. One-way analyses of variance (one-way ANOVA) were carried out with an SAS software package for computer-based statistical analysis, and Duncan's test was used to determine if there was any difference between the different treatments.

### Experiment 1: Preparation of a Streptococcus Thermophiles ST7 Fermentation Product Composition

The Streptococcus salivarius subsp. thermophilus ST7 disclosed herein was cultivated in a culture medium containing 1%-2% glucose, 1%-2% peptone, and 0.01%-0.08% magnesium sulfate, among other ingredients. Once reaching the stage of logarithmic growth, the Streptococcus salivarius subsp. thermophilus ST7 was inoculated into a principal-fermentation culture medium and then allowed to grow and ferment under predetermined fermentation conditions in order to produce a fermentation product. The principal-fermentation culture medium contained 5%-15% glucose, 2%-6% peptone, and 0.01%-0.08% magnesium sulfate, among other ingredients. The predetermined fermentation conditions included a fermentation pH value of 4.0-6.0, a fermentation temperature of 35-40° C., and a fermentation time of 10-24 hours.

The fermentation product went through such processes as condensation and drying, and a Streptococcus thermophiles ST7 fermentation product composition was obtained as a result (the bacteria count being 4×10¹⁰ cell/g). The drying process may involve spray drying, low-temperature drying, freeze drying, or other drying techniques well known to a person skilled in the art.

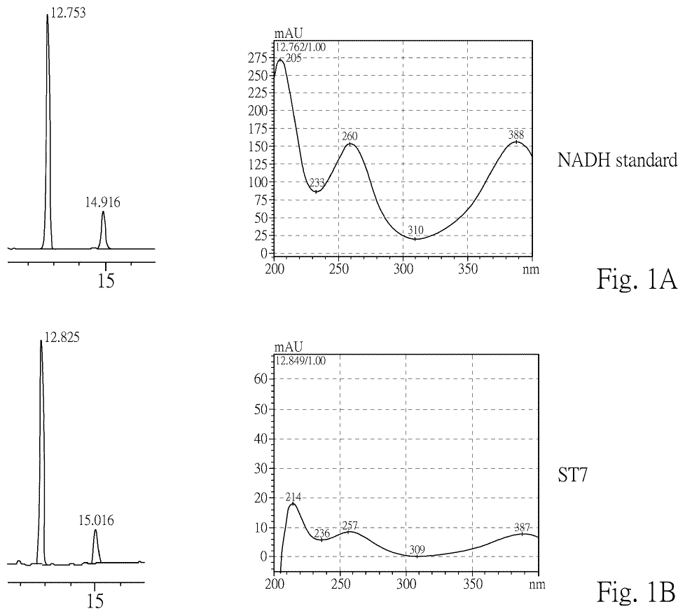

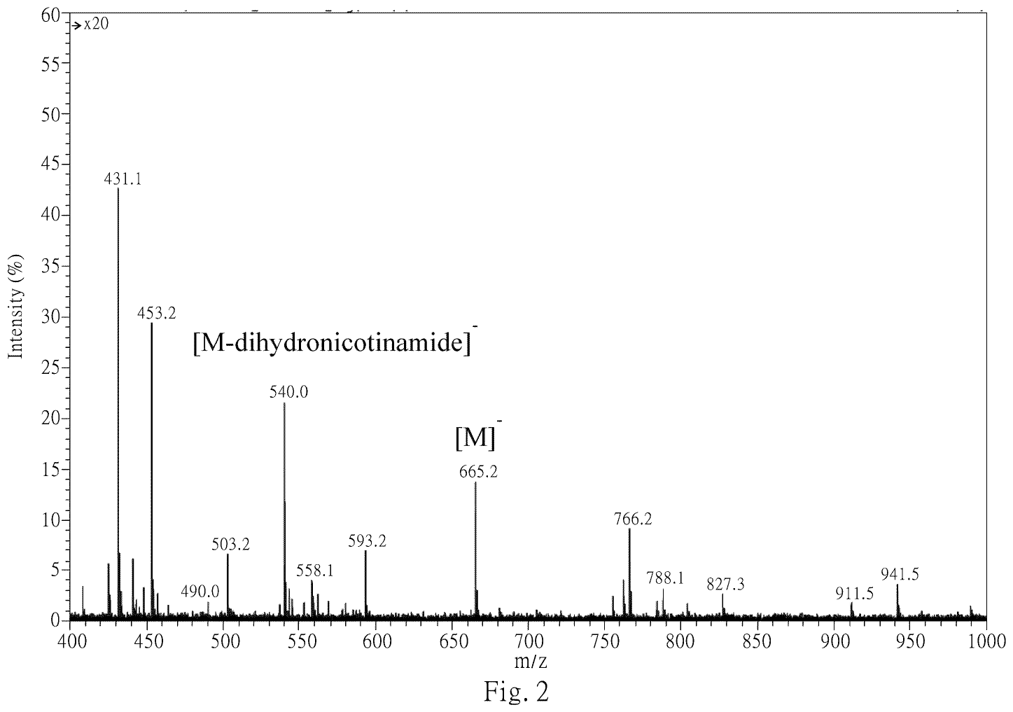

The Streptococcus thermophiles ST7 fermentation product composition (1 g) and an NADH standard (5 mg) were each mixed with water to make a 10 mL aqueous solution. Each aqueous solution (250 μL) was added with deionized water (100 μL), potassium hydroxide (150 μL, 1.3 M), and acetophenone (100 μL, 20%), mixed well, then put in an iced water bath, added with methanoic acid (400 μL), and then allowed to stand at room temperature in order for reactions to take place. After that, the aqueous solutions were filtered and subjected to an HPLC analysis. The analysis conditions were as follows: the eluent was acetonitrile, the phosphate buffered solution (0.1 M, pH 6.5) was a 10:90 mixed solution, the column was Supelco C18 (250×4.6, 5 μm), and the wavelength used in the analysis was 382 nm. The HPLC analysis results are shown in FIG. 1A and FIG. 1B. In addition, the Streptococcus thermophiles ST7 fermentation product composition was subjected to an ESI-MS analysis, whose result is shown in FIG. 2.

It can be known from the results in FIG. 1A and FIG. 1B that the Streptococcus thermophiles ST7 fermentation product composition disclosed herein contained the fermentation product, and that the fermentation product contained NADH. It can be further known from the result in FIG. 2 that the molecular formula of this NADH was C₂₁H₂₉N₇O₁₄P₂⁻, with the mass-to-charge ratio of [M]⁻ being 665.2.

### Experiment 2: Cell Culture

C2C12 myoblasts (deposit number: BCRC NO. 60083) were inoculated at a density of 3×10⁵ cells/well into the Dulbecco's modified Eagle's medium (DMEM) in a 60 mm petri dish, wherein the DMEM contained 10% penicillin-streptomycin solution. The C2C12 myoblasts were cultured at 37° C. for 1 day for cell differentiation. Following that, the cell culture medium was changed to a DMEM added with 2% horse serum and was renewed every day. After 5 days of differentiation, the C2C12 myoblasts were ready for use in the following cell experiments.

### Experiment 3: Cell Experiment (1)

Some of the C2C12 myoblasts cultured in experiment 2 were taken and added with different substances separately. The substances included the drug Metformin (2.5 mM); a de Man, Rogosa, and Sharpe agar (MRS); a supernatant of the Streptococcus thermophiles ST7 fermentation product disclosed herein; a supernatant of a fermentation product of Lactobacillus paracasei MC1-40 (deposit number: BCRC 911125 or DSM 34254); and a supernatant of a fermentation product of Lactobacillus paracasei LCW23 (deposit number: CGMCC 3247). The final concentration of each cell group was 1%. The cells were then cultured for another 24 hours. The supernatant of the Streptococcus thermophiles ST7 fermentation product was the filtrate obtained by filtering a solution prepared by re-dissolving the Streptococcus thermophiles ST7 fermentation product composition in experiment 1. The supernatant of the Lactobacillus paracasei MC1-40 fermentation product and the supernatant of the Lactobacillus paracasei LCW23 fermentation product were prepared in the same way as the supernatant of the Streptococcus thermophiles ST7 fermentation product.

The Lactobacillus paracasei MC1-40 is deposited at German Collection of Microorganism Cell Cultures (DSMZ) (the deposit date being Apr. 28, 2022, and the deposit number being DSM 34254) and at the Food Industry Research and Development Institute of Taiwan (the deposit date being Apr. 25, 2022, and the deposit number being BCRC 911125).

The Lactobacillus paracasei LCW23 is deposited at the China General Microbiological Culture Collection Center (CGMCC), the deposit date being Aug. 21, 2009, and the deposit number being CGMCC 3247.

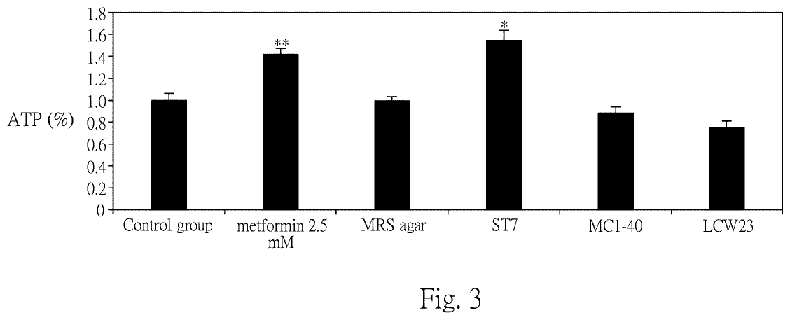

Once cultivation was completed, the C2C12 myoblasts in each group were collected, washed with a phosphate buffered solution, then collected with trypsin-EDTA, and centrifuged. The cell pellets obtained for each group were re-dissolved in a tris(hydroxymethyl)aminomethane buffer (tris buffer) to break up the pellets, before the supernatant was collected and analyzed with an ATP colorimetric assay kit (Elabscience, catalog no. E-BC-K157-S) in order to quantify the ATP content of the cells at an absorption wavelength of 636 nm. The analysis results are shown in FIG. 3, in which ST7, MC1-40, and LCW23 denote the supernatants collected from the fermentation products of their respective strains.

According to the results in FIG. 3, the Streptococcus thermophiles ST7 fermentation product composition disclosed herein was indeed capable of increasing the ATP content of the C2C12 myoblasts. It is therefore reasonable to infer that the C2C12 myoblasts disclosed herein can increase muscle strength and thereby alleviate or improve sarcopenia and its symptoms.

### Experiment 4: Cell Experiment (2)

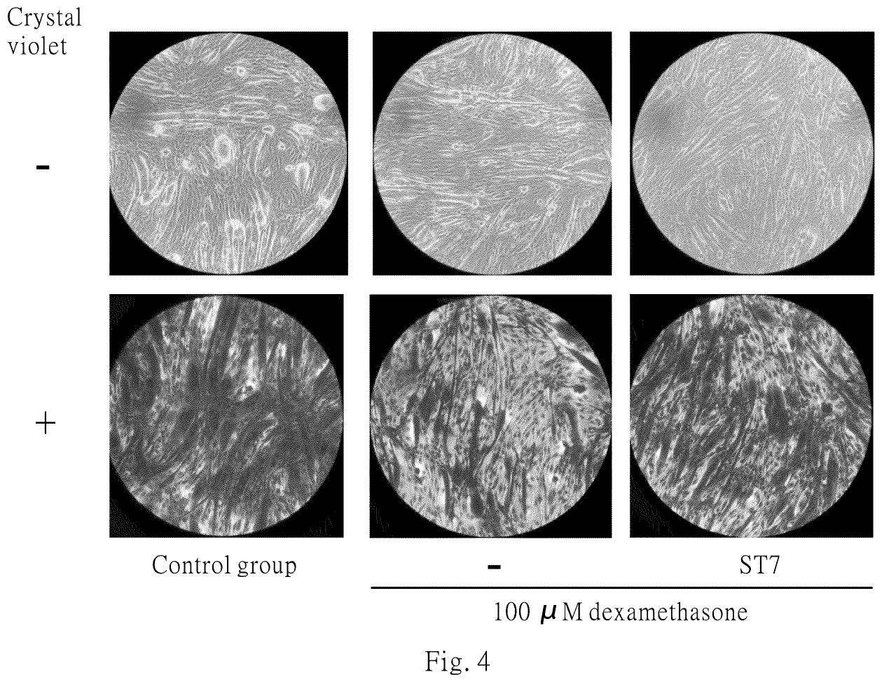

Some of the C2C12 myoblasts cultured in experiment 2 were taken and added separately with a DMEM (control group), 100 μM dexamethasone, and 100 μM dexamethasone and a supernatant of the Streptococcus thermophiles ST7 fermentation product disclosed herein. The final concentration of each cell group was 1%. The cells were then cultured for another 48 hours. After that, the cells were stained with crystal violet to facilitate observation of the death of cells in each cell group. The observation results are shown in FIG. 4.

Dexamethasone is a reagent that can induce myotube atrophy. Therefore, as shown in FIG. 4, the C2C12 myoblasts cultured in the medium added only with dexamethasone showed more notable atrophy and death than those in the control group. By contrast, the C2C12 myoblasts cultured in the medium added with dexamethasone and the supernatant of the Streptococcus thermophiles ST7 fermentation product disclosed herein showed a significant improvement in cell death.

The results in FIG. 4 show that the Streptococcus thermophiles ST7 fermentation product composition disclosed herein was effective in improving myotube atrophy and death of the C2C12 myoblasts. From this it can be inferred that the Streptococcus thermophiles ST7 fermentation product composition disclosed herein can delay muscle loss or muscle disability and is hence effective in treating or preventing sarcopenia and enhancing an individual's sports performance.

### Experiment 5: Animal Test

Twenty-four six-week-old male Institute of Cancer Research (ICR) mice were fed for two weeks and then randomly divided into three groups, each including eight mice. During the following four weeks, the Streptococcus thermophiles ST7 fermentation product composition disclosed herein was administered (or not administered) to the mice in each group as follows, with the mice allowed to eat feed (Chow 5001) and water freely, raised at a temperature of 24±2° C. and a humidity of 65±5%, and kept under a 12-hour light, 12-hour dark cycle:
Group 1: This group of mice was not given the Streptococcus thermophiles ST7 fermentation product composition disclosed herein.
Group 2: This group of mice was given the Streptococcus thermophiles ST7 fermentation product composition disclosed herein at a low dose of 21 mg/Kg/day.
Group 3: This group of mice was given the Streptococcus thermophiles ST7 fermentation product composition disclosed herein at a high dose of 205 mg/Kg/day.

The body weights of the mice in each group were measured during the test, and the measurement results are shown in Table 1.

TABLE 1
Body weights of the mice in each group during the test

| | Initial body | | | | | | |
|---|---|---|---|---|---|---|---|
| | weight (g) | Week 1 (g) | Week 2 (g) | Week 3 (g) | Week 4 (g) | Week 5 (g) | Week 6 (g) |
| Group 1 | 29.78 ± 0.35 | 31.48 ± 0.41 | 32.99 ± 0.49 | 34.54 ± 0.49 | 35.99 ± 0.51 | 37.14 ± 0.59 | 38.48 ± 0.37 |
| Group 2 | 29.74 ± 0.71 | 31.64 ± 0.88 | 33.11 ± 1.10 | 34.37 ± 1.14 | 35.59 ± 1.04 | 36.92 ± 1.09 | 38.05 ± 1.10 |
| Group 3 | 29.78 ± 0.77 | 31.08 ± 0.87 | 32.28 ± 0.77 | 33.79 ± 0.50 | 35.06 ± 0.57 | 36.45 ± 0.58 | 37.75 ± 0.58 |

It can be known from the results in Table 1 that there was no difference in body weight between the groups before the test, and that the body weights of the animals in each group increased steadily after the test started, indicating that the Streptococcus thermophiles ST7 fermentation product composition disclosed herein had neither side effect on the animals nor adverse effect on the growth of the animals.

### Experiment 6: Biochemical Test of Blood

Once the test of experiment 5 was completed, the mice were sacrificed, and their blood was collected from the hearts and centrifuged at 4° C. and 15000×g for 15 minutes. After that, the serum was collected for an analysis of the liver damage indices AST (aspartate aminotransferase) and ALT (alanine aminotransferase) by an automatic blood analyzer (Hitachi 7060, Hitachi, Tokyo, Japan). The analysis results are shown in Table 2.

TABLE 2
Biochemical analysis results of the blood of the mice in each group

| Index | AST (U/L) | ALT (U/L) |
|---|---|---|
| Group 1 | 74.75 ± 3.01 | 41.75 ± 3.20 |
| Group 2 | 75.00 ± 5.71 | 41.13 ± 5.82 |
| Group 3 | 74.88 ± 23.85 | 41.25 ± 4.59 |

It can be known from the results in Table 2 that there was no significant difference in the values of the liver damage indices between the mouse groups, indicating that the Streptococcus thermophiles ST7 fermentation product composition disclosed herein did not affect the liver functions of the animals.

### Experiment 7: Forelimb Grip Strength Test

On the 29^{th} day of the test of experiment 5 (i.e., after the Streptococcus thermophiles ST7 fermentation product composition disclosed herein had been administered for four weeks), a forelimb grip strength test was performed on the mice in each group, and relative grip strength calculated. The test results are shown in Table 3.

TABLE 3
Forelimb grip strength test results of the mice in each group

| | Grip strength (g) | Relative grip strength (%) |
|---|---|---|
| Group 1 | 112.00 ± 4.84 | 305.20 ± 18.63 |
| Group 2 | 126.00 ± 7.54 | 339.77 ± 25.54 |
| Group 3 | 131.38 ± 11.93 | 356.88 ± 39.23 |

It can be known from the results in Table 3 that the absolute forelimb grip strength of the mice in groups 2 and 3 was 1.13 times and 1.17 times as great as that of the mice in group 1 respectively, and that the relative forelimb grip strength of the mice in groups 2 and 3 was 1.13 times and 1.17 times as great as that of the mice in group 1 respectively.

According to the results in Table 3, administering an effective amount of the Streptococcus thermophiles ST7 fermentation product composition disclosed herein to an individual was indeed able to increase the individual's grip strength, and that the increase in grip strength was not affected by the individual's body weight. It can therefore be inferred that the Streptococcus thermophiles ST7 fermentation product composition disclosed herein is effective in improving or alleviating sarcopenia and the related symptoms and enhancing an individual's sports performance.

### Experiment 8: Endurance Test

One week before the swimming test, each group of mice went through a swimming adaptation process (with the water temperature being 27±1° C.). On the 31^{st} day of the test, the mice in each group were made to swim under load (5% of their respective body weights) to exhaustion. More specifically, the mice were forced to swim by being put into a transparent water tank filled with water (the diameter of the tank being 15 cm, the water depth being 20 cm, and the water temperature being 27±1° C.). The swimming time to exhaustion of the mice in each group was measured and is shown in Table 4. The mice were fasted for 12 hours before swimming, and the Streptococcus thermophiles ST7 fermentation product composition administered on the swimming day was administered 30 minutes before the swimming test.

TABLE 4
Swimming time to exhaustion of the mice in each group

| | Swimming time to exhaustion (minute) |
|---|---|
| Group 1 | 3.50 ± 0.25 |
| Group 2 | 8.92 ± 0.53 |
| Group 3 | 9.43 ± 0.32 |

It can be known from the results in Table 4 that the swimming time to exhaustion of the mice in groups 2 and 3 was 2.55 times and 2.69 times as long as that of the mice in group 1 respectively. The results show that administration of the Streptococcus thermophiles ST7 fermentation product composition disclosed herein was able to increase the ATP content of muscle cells and thereby enhance muscle abilities and consequently the animals' sports performance.

### Experiment 9: Analysis of Blood Lactic Acid Concentration

In the course of experiment 8, the blood lactic acid concentrations of the mice in each group were measured at three time points: before swimming, after a 10-minute rest after swimming, and after a 20-minute rest after swimming. After-exercise to before-exercise blood lactic acid concentration ratios were also calculated. The measurements and calculation results are shown in Table 5.

TABLE 5
Blood lactic acid concentrations of the mice in each group before and after
exercise

| | Before | After 10- | After 20- | | |
|---|---|---|---|---|---|
| | swimming | minute rest | minute rest | | Removal rate = |
| Time point | (A) | after swimming | after swimming | Yield = B/A | (B − C)/B |
| Group 1 | 3.53 ± 0.34 | 8.41 ± 0.39 | 7.75 ± 0.29 | 2.40 ± 0.26 | 0.08 ± 0.06 |
| Group 2 | 3.59 ± 0.26 | 6.31 ± 0.42 | 5.45 ± 0.42 | 1.76 ± 0.17 | 0.13 ± 0.05 |
| Group 3 | 3.62 ± 0.41 | 5.70 ± 0.59 | 4.85 ± 0.32 | 1.60 ± 0.31 | 0.14 ± 0.13 |

It can be known from the results in Table 5 that before swimming, there was no significant difference in blood lactic acid concentration between the mouse groups; that 10 minutes after swimming, the blood lactic acid concentrations of the mice in groups 1, 2, and 3 were 8.41±0.39, 6.31±0.42, and 5.70±0.59 mmol/L respectively; and that 20 minutes after swimming, the blood lactic acid concentrations of the mice in groups 1, 2, and 3 were 7.75±0.29, 5.45±0.42, and 4.85±0.32 mmol/L respectively. In addition, the blood lactic acid removal rate of each group was calculated by dividing the difference between the corresponding blood lactic acid concentration at the time point 10 minutes after swimming and the corresponding blood lactic acid concentration 20 minutes after swimming by the corresponding blood lactic acid concentration at the time point 10 minutes after swimming. The lactic acid removal rates of the mice in groups 1, 2, and 3 were 0.08±0.06, 0.13±0.05, and 0.14±0.13 respectively.

It can be inferred from the results in Table 5 that administering an effective amount of the Streptococcus thermophiles ST7 fermentation product composition disclosed herein to an individual before exercise can effectively reduce the lactic acid produced after the exercise and enhance the individual's ability to remove or metabolize the lactic acid in the body, thereby reducing the feeling of fatigue, promoting muscle restoration, enhancing the individual's sports performance, and helping to prevent or improve sarcopenia.

### Experiment 10: Analysis of BUN Content and Creatine Kinase Content

On the 35^{th} day of the test of experiment 5, and 30 minutes after the Streptococcus thermophiles ST7 fermentation product composition disclosed herein was administered, the mice in each group were made to swim without load for 90 minutes (the water temperature being 30° C.), and blood was collected from the mice after a 60-minute rest and subjected to an analysis of BUN content and creatine kinase (CK) content. The analysis results are shown in Table 6.

TABLE 6
BUN content and CK activity analysis of each mouse group

| | BUN before | BUN after 60-minute | CK after 60-minute |
|---|---|---|---|
| | swimming | rest after swimming | rest after swimming |
| Index | (mg/dL) | (mg/dL) | (mg/dL) |
| Group 1 | 22.15 ± 0.59 | 42.59 ± 1.89 | 1934.88 ± 105.06 |
| Group 2 | 22.76 ± 0.72 | 36.33 ± 4.25 | 1632.00 ± 285.39 |
| Group 3 | 22.35 ± 2.27 | 35.74 ± 3.16 | 1664.50 ± 163.47 |

It can be known from the results in Table 6 that the after-swimming BUN concentrations of groups 1, 2, and 3 were 42.59±1.89, 36.33±4.25, and 35.74±3.16 mg/dL respectively. A further analysis reveals that the BUN contents of the mice in groups 2 and 3 were significantly lower than that of the mice in group 1 by 14.7% and 16.1% respectively.

As to the change in activity of blood CK, the CK contents of groups 1, 2, and 3 were 1934.88±105.06, 1632.00±285.39, and 1664.50±163.47 U/L respectively, and a further analysis reveals that the blood CK activity of the mice in groups 2 and 3 was significantly lower than that of the mice in group 1 by 15.7% and 14.0% respectively.

The foregoing results prove that administering an effective amount of the Streptococcus thermophiles ST7 fermentation product composition disclosed herein to an individual is helpful in reducing the BUN concentration and CK activity after exercise. In other words, the Streptococcus thermophiles ST7 fermentation product composition disclosed herein can help repair muscle damage caused by exercise or physical work and is therefore effective in enhancing an individual's sports performance and willingness to exercise and hence in preventing or alleviating sarcopenia.

### Experiment 11: Analysis of the Glycogen Contents of Liver and Muscles

After a 90-minute no-load swimming test (as described above in relation to experiment 10), the mice in each group were allowed to rest for two days (i.e., till the 37^{th} day of the test of experiment 5) and then fed with the Streptococcus thermophiles ST7 fermentation product composition disclosed herein. The mice were sacrificed 30 minutes after being fed, and the livers and leg muscles of the mice were taken and subjected to a glycogen content analysis. A commercially available glycogen standard (Glycogen Sigma) was used to plot the calibration curve, and the changes in the amounts of glycogen stored in the livers and muscle tissues of the animals in each group were calculated. The analysis results are shown in TABLE 7.

TABLE 7
Glycogen contents of the livers and muscles of the mice in each group

| | Glycogen content of liver | Glycogen content of muscles |
|---|---|---|
| Index | (mg/g) | (mg/g) |
| Group 1 | 12.21 ± 0.96 | 0.97 ± 0.24 |
| Group 2 | 16.88 ± 2.21 | 1.07 ± 0.37 |
| Group 3 | 20.64 ± 2.29 | 1.36 ± 0.53 |

According to the results in Table 7, the glycogen contents of the livers of the mice in groups 1, 2, and 3 were 12.21±0.96, 16.88±2.21, and 20.64±2.29 mg/g liver, respectively. A further analysis of the data reveals that the glycogen contents of the livers of the mice in groups 2 and 3 were significantly higher than that of the mice in group 1, or were 1.38 times and 1.69 times as high as that of the mice in group 1 respectively, to be exact. In addition, the glycogen contents of the muscles of the mice in groups 1, 2, and 3 were 0.97±0.24, 1.07±0.37, and 1.36±0.53 mg/g muscle, respectively, and a further analysis of the data reveals that the glycogen content of the muscles of the mice in group 3 was significantly higher than, or 1.40 times as high as, that of the mice in group 1.

The results in Table 7 prove that administering an effective amount of the Streptococcus thermophiles ST7 fermentation product composition disclosed herein to an individual can help increase the glycogen contents of the individual's liver and muscles and thereby enhance or improve muscle abilities and muscle mass, which in turn contributes to enhancing the individual's sports performance and alleviating sarcopenia and its symptoms.

1. A method for enhancing sports performance and treating or preventing sarcopenia with a Streptococcus thermophilus ST7 fermentation product composition, comprising the step of administering an effective amount of the Streptococcus thermophilus ST7 fermentation product composition to an individual to increase an adenosine triphosphate (ATP) content of muscle cells, to increase a glycogen content of muscle cells and of liver cells, to reduce blood lactic acid content and blood urea nitrogen (BUN) concentration, and to increase a repair ability of muscle cells, thereby enhancing the individual's sports performance and alleviating or preventing sarcopenia and symptoms thereof;
wherein:
the Streptococcus thermophilus ST7 fermentation product composition comprises a Streptococcus thermophilus ST7 fermentation product;
the Streptococcus thermophilus ST7 fermentation product is obtained by fermentation of Streptococcus salivarius subsp. thermophilus ST7; and
the Streptococcus salivarius subsp. thermophilus ST7 is deposited at German Collection of Microorganism Cell Cultures (DSMZ), the deposit date being Apr. 28, 2022, and the deposit number being DSM 34255.

2. The method of claim 1, wherein the Streptococcus thermophilus ST7 fermentation product comprises at least one component selected from the group consisting of the Streptococcus salivarius subsp. thermophilus ST7, inactivated bacteria of the Streptococcus salivarius subsp. thermophilus ST7, metabolites of the Streptococcus salivarius subsp. thermophilus ST7, and NADH (nicotinamide adenine dinucleotide in reduced form).

3. The method of claim 1, wherein the Streptococcus thermophilus ST7 fermentation product comprises inactivated bacteria of the Streptococcus salivarius subsp. thermophilus ST7, metabolites of the Streptococcus salivarius subsp. thermophilus ST7, and NADH (nicotinamide adenine dinucleotide in reduced form).

4. The method of claim 1, wherein a said symptom of sarcopenia is the individual having low grip strength.

5. The method of claim 1, wherein the individual belongs to a population at high risk of sarcopenia.
